(19) United States
(12) Patent Application Publication
(10) Pub. No.: US 2021/0338200 A1
(43) Pub. Date: Nov. 4, 2021

(54) ULTRASOUND IMAGING APPARATUS, OPERATING METHOD OF ULTRASOUND IMAGING APPARATUS, AND COMPUTER-READABLE RECORDING MEDIUM

(71) Applicant: OLYMPUS CORPORATION, Tokyo (JP)

(72) Inventor: Junichi ICHIKAWA, Tokyo (JP)

(73) Assignee: OLYMPUS CORPORATION, Tokyo (JP)

(21) Appl. No.: 17/378,940

(22) Filed: Jul. 19, 2021

Related U.S. Application Data
(63) Continuation of application No. PCT/JP2019/003200, filed on Jan. 30, 2019.

(51) Int. Cl. A61B 8/12; G01S 15/89; A61B 8/08; G16H 40/67; G16H 50/70; G16H 30/20; G16H 30/40

(52) U.S. Cl. CPC A61B 8/12; G01S 15/8959; G01S 15/8977; G16H 30/40; G16H 40/67; G16H 50/70; G16H 30/20; A61B 8/5207

(57) ABSTRACT

An ultrasound imaging apparatus includes: a processor; and a storage. The storage is configured to store first-type reference data corresponding to a first observation target and second-type reference data corresponding to a second observation target. The processor is configured to transmit, to an ultrasound probe, a signal for making the ultrasound probe transmit an ultrasound wave to an observation target, receive an echo signal, perform frequency analysis based on the echo signal to calculate a frequency spectrum, obtain reference data, correct the frequency spectrum using the reference data, calculate a feature based on the corrected frequency spectrum, when the observation target is the first observation target, obtain the first-type reference data as the reference data, and when the observation target is the second observation target, obtain the second-type reference data as the reference data.

### CROSS-REFERENCE TO RELATED APPLICATION

This application is a continuation of International Application No. PCT/JP2019/003200, filed on Jan. 30, 2019, the entire contents of which are incorporated herein by reference.

### BACKGROUND

### 1. Technical Field

The present disclosure is related to an ultrasound imaging apparatus that observes the anatomy of an observation target using ultrasound waves; is related to an operating method of the ultrasound imaging apparatus; and is related to an a computer-readable recording medium.

### 2. Related Art

In order to observe the characteristics of an observation target such as biological tissues or a biomaterial, sometimes ultrasound waves are used. More particularly, ultrasound waves are transmitted to the observation target; predetermined signal processing is performed with respect to the ultrasound wave echo reflected from the observation target; and information regarding the characteristics of the observation target is obtained (for example, refer to Japanese Patent Application Laid-open No. 2013-166059). In Japanese Patent Application Laid-open No. 2013-166059, a frequency spectrum is calculated by analyzing the frequencies of the ultrasound waves received from the observation target; and the frequency spectrum is corrected using a reference spectrum. The reference spectrum is calculated based on the frequencies of the ultrasound waves received from a reference reflector.

### SUMMARY

In some embodiments, an ultrasound imaging apparatus includes: a processor; and a storage. The storage is configured to store first-type reference data corresponding to a first observation target, and second-type reference data corresponding to a second observation target. The processor is configured to transmit, to an ultrasound probe, a signal for making the ultrasound probe transmit an ultrasound wave to an observation target, receive an echo signal representing an electrical signal obtained by conversion of an ultrasound wave received by the ultrasound probe, perform frequency analysis based on the echo signal to calculate a frequency spectrum, obtain reference data, correct the frequency spectrum using the reference data, calculate a feature based on the corrected frequency spectrum, when the observation target is the first observation target, obtain the first-type reference data as the reference data, and when the observation target is the second observation target, obtain the second-type reference data as the reference data.

In some embodiments, provided is an operating method of an ultrasound imaging apparatus configured to generate an ultrasound image based on an ultrasound signal obtained by an ultrasound probe which includes an ultrasound transducer for transmitting an ultrasound wave to an observation target and receiving an ultrasound wave reflected from the observation target. The operating method includes: transmitting, to the ultrasound probe, a signal for making the ultrasound probe transmit an ultrasound wave to an observation target, receiving an echo signal representing an electrical signal obtained by conversion of an ultrasound wave received by the ultrasound probe; performing frequency analysis based on the echo signal to calculate a frequency spectrum; obtaining first-type reference data when the observation target is a first observation target, obtaining second-type reference data when the observation target is a second observation target, correcting the frequency spectrum using the obtained first-type reference data or the obtained second-type reference data; and calculating a feature based on the corrected frequency spectrum.

In some embodiments, provided is a non-transitory computer-readable recording medium with an executable program stored thereon. The program causes an ultrasound imaging apparatus configured to generate an ultrasound image based on an ultrasound signal obtained by an ultrasound probe which includes an ultrasound transducer for transmitting an ultrasound wave to an observation target and receiving an ultrasound wave reflected from the observation target, to execute: transmitting, to the ultrasound probe, a signal for making the ultrasound probe transmit an ultrasound wave to an observation target, receiving an echo signal representing an electrical signal obtained by conversion of an ultrasound wave received by the ultrasound probe; performing frequency analysis based on the echo signal to calculate a frequency spectrum; obtaining first-type reference data when the observation target is a first observation target, obtaining second-type reference data when the observation target is a second observation target, correcting the frequency spectrum using the obtained first-type reference data or the obtained second-type reference data; and calculating a feature based on the corrected frequency spectrum.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an ultrasound imaging system that includes an ultrasound imaging apparatus according to a first embodiment of the disclosure;

FIG. 2 is a diagram that schematically illustrates an example of a frequency spectrum calculated based on the ultrasound waves coming from a scattering body;

FIG. 3A is a diagram illustrating an example of the scattering body;

FIG. 3B is a diagram for explaining an example of reference data that is used in a correction operation for correcting the frequency spectrum as performed in the ultrasound imaging apparatus according to the first embodiment;

FIG. 4A is a diagram illustrating an example of the scattering body;

FIG. 4B is a diagram for explaining an example of reference data that is used in a correction operation for correcting the frequency spectrum as performed in the ultrasound imaging apparatus according to the first embodiment;

FIG. 5 is a diagram illustrating an example of a post-correction frequency spectrum obtained as a result of the correction performed by a spectrum correction unit of the ultrasound imaging apparatus according to the first embodiment of the disclosure;

FIG. 6 is a diagram for explaining about a post-correction frequency spectrum obtained by performing correction using reference data that does not correspond to any scattering body;

FIG. 7 is a diagram for explaining about a post-correction frequency spectrum obtained by performing correction using reference data that corresponds to a scattering body;

FIG. 8 is a flowchart for explaining an overview of the operations performed by the ultrasound imaging apparatus according to the first embodiment of the disclosure;

FIG. 9 is a diagram that schematically illustrates an exemplary display of a feature image in a display device of the ultrasound imaging apparatus according to the first embodiment of the disclosure;

FIG. 10 is a block diagram illustrating a configuration of an ultrasound imaging system that includes an ultrasound imaging apparatus according to a second embodiment of the disclosure;

FIG. 11 is a diagram for explaining an organ determination operation performed in an ultrasound imaging apparatus according to a first modification example of the second embodiment of the disclosure; and

FIG. 12 is a diagram for explaining an organ determination operation performed in an ultrasound imaging apparatus according to a second modification example of the second embodiment of the disclosure.

### DETAILED DESCRIPTION

Exemplary embodiments of the disclosure are described below with reference to the accompanying drawings.

### First Embodiment

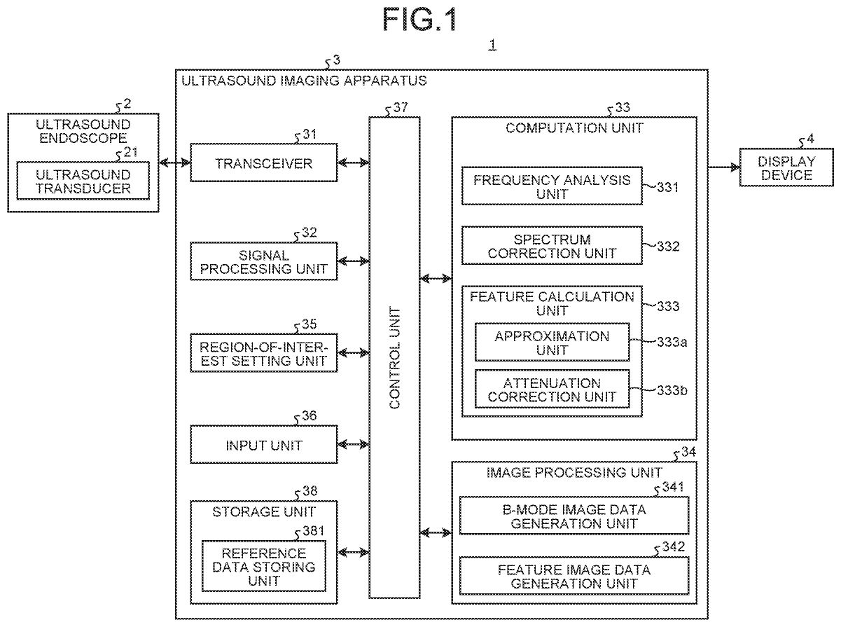

FIG. 1 is a block diagram illustrating a configuration of an ultrasound imaging system 1 that includes an ultrasound imaging system 3 according to a first embodiment of the disclosure. The ultrasound imaging system 1 illustrated in FIG. 1 includes: an ultrasound endoscope 2 (an ultrasound probe) that transmits ultrasound waves to a subject representing the observation target and receives the ultrasound waves reflected from the subject; the ultrasound imaging apparatus 3 that generates ultrasound images based on the ultrasound wave signals obtained by the ultrasound endoscope 2; and a display device 4 that displays the ultrasound images generated by the ultrasound imaging apparatus 3.

The ultrasound endoscope 2 includes an ultrasound transducer 21 that converts electrical pulse signals, which are received from the ultrasound imaging apparatus 3, into ultrasound pulses (acoustic pulses) and irradiates the subject with the acoustic pulses; and then converts the ultrasound wave echo reflected from the subject into electrical echo signals expressed in terms of voltage variation, and outputs the electrical echo signals. The ultrasound transducer 21 includes piezoelectric elements arranged in a one-dimensional manner (linear manner) or a two-dimensional manner, and transmits and receives ultrasound waves using the piezoelectric elements. The ultrasound transducer 21 can be a convex transducer, a linear transducer, or a radial transducer.

The ultrasound endoscope 2 usually includes an imaging optical system and an imaging device. The ultrasound endoscope 2 is inserted into the alimentary tract (the esophagus, the stomach, the duodenum, or the large intestine) of the subject or into a respiratory organ (the trachea or the bronchus) of the subject, and is capable of taking images of the alimentary canal or the respiratory organ and the surrounding organs (such as the pancreas, the gallbladder, the biliary duct, the biliary tract, the lymph node, the mediastinal organs, and the blood vessels). Moreover, the ultrasound endoscope 2 includes a light guide for guiding an illumination light that is thrown onto the subject at the time of imaging. The light guide has the front end thereof reaching the front end of the insertion portion of the ultrasound endoscope 2 that is to be inserted into the subject, and has the proximal end thereof connected to a light source device that generates the illumination light. Meanwhile, instead of using the ultrasound endoscope 2, an ultrasound probe can be used that does not include an imaging optical system and an imaging device.

The ultrasound imaging apparatus 3 includes: a transceiver 31 that is electrically connected to the ultrasound endoscope 2, that transmits transmission signals (pulse signals) of high-voltage pulses to the ultrasound transducer 21 based on predetermined waveforms and transmission timings, and that receives echo signals representing electrical reception signals from the ultrasound transducer 21 and generates and outputs data of digital radio frequency (RF) signals (hereinafter called RF data); a signal processing unit 32 that generates digital B-mode reception data based on the RF data received from the transceiver 31; a computation unit 33 that performs predetermined computations with respect to the RF data received from the transceiver 31; an image processing unit 34 that generates a variety of image data; a region-of-interest setting unit 35 that sets a region of interest regarding the image data generated by the image processing unit 34; an input unit (input device) 36 that is implemented using a user interface such as a keyboard, a mouse, or a touch-sensitive panel and that receives input of a variety of information; a control unit 37 that controls the entire ultrasound imaging system 1; and a storage unit 38 that is used to store a variety of information required in the operation of the ultrasound imaging apparatus 3.

The transceiver 31 performs processing such as filtering with respect to the received echo signals; performs A/D conversion to generate RF data in the time domain; and outputs the RF data to the signal processing unit 32 and the computation unit 33. At that time, the transceiver 31 can perform amplitude correction according to the reception depth. Meanwhile, if the ultrasound endoscope 2 is configured to perform electronic scanning of the ultrasound transducer 21 in which a plurality of elements is arranged in an array, the transceiver 31 has a multichannel circuit for beam synthesis corresponding to the plurality of elements.

The frequency band of the pulse signals, which are transmitted by the transceiver 31, preferably has a broad spectrum that substantially covers the linear response frequency bandwidth of electroacoustic conversion of pulse signals into ultrasound pulses in the ultrasound transducer 21. As a result, at the time of performing approximation of the frequency spectrum (explained later), it becomes possible to perform the approximation with high accuracy.

The transceiver 31 transmits various controls signals, which are output by the control unit 37, to the ultrasound endoscope 2; and also has the function of receiving a variety of information that contains an identification ID from the ultrasound endoscope 2 and transmitting the information to the control unit 37.

The signal processing unit 32 performs known processing such as bandpass filtering, envelope detection, and logarithmic conversion with respect to the RF data, and generates digital B-mode reception data. In the logarithmic conversion, the common logarithm is taken for the quantity obtained by dividing the RF data by a reference voltage V_{c}, and the common logarithm is expressed as a digital value. Then, the signal processing unit 32 outputs the B-mode reception data to the image processing unit 34. The signal processing unit 32 is configured using a general-purpose processor such as a central processing unit (CPU); or using a dedicated processor in the form of an arithmetic circuit such as a field programmable gate array (FPGA) or an application specific integrated circuit (ASIC) having specific functions.

The computation unit 33 includes: a frequency analysis unit 331 that performs frequency analysis by performing fast Fourier transform (FFT) with respect to the RF data generated by the transceiver 31, and calculates a frequency spectrum; a spectrum correction unit 332 that corrects the frequency spectrum, which is calculated by the frequency analysis unit 331, using reference data stored in the storage unit 38; and a feature calculation unit 333 that calculates a feature of the frequency spectrum corrected by the spectrum correction unit 332. The computation unit 33 is configured using a general-purpose processor such as a CPU; or using a dedicated processor in the form of an arithmetic circuit such as an FPGA or an ASIC having specific functions.

The frequency analysis unit 331 samples the RF data of each sound ray (i.e., line data), which is generated by the transceiver 31, at predetermined time intervals and generates sets of sample data. Then, the frequency analysis unit 331 performs FFT with respect to the sample data group, and calculates the frequency spectrum at a plurality of positions in the RF data (data positions). Herein, the term “frequency spectrum” implies “the frequency distribution having the intensity at a particular reception depth z” as obtained by performing FFT with respect to the sample data group. Moreover, the term “intensity” implies parameters such as the voltage of the echo signals, the electrical power of the echo signals, the sound pressure of the ultrasound wave echo, and the acoustic energy of the ultrasound wave echo; or implies the amplitude of those parameters, the time division value of those parameters, or a combination of those parameters.

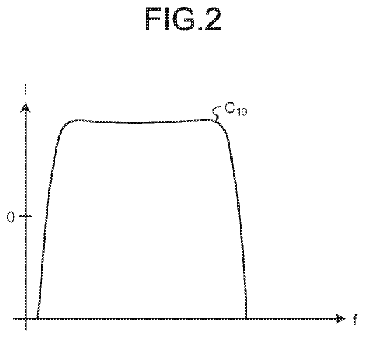

FIG. 2 is a diagram that schematically illustrates an example of the frequency spectrum calculated based on the ultrasound waves coming from a scattering body. The frequency obtaining unit 331 obtains for example, a frequency spectrum C₁₀ illustrated in FIG. 2. The frequency spectrum C₁₀ corresponds to a scattering body Q_{A} (explained later). In FIG. 2, the horizontal axis represents a frequency f. Moreover, in FIG. 2, the vertical axis represents a common logarithm (digital expression) I of the quantity obtained by dividing an intensity I₀ by a reference intensity I_{c} (a constant number) (i.e., (I=10 log₁₀(I₀/I_{c}) holds true). Meanwhile, in the first embodiment, curved lines and straight lines are made of a set of discrete points.

Generally, when biological tissues represent the subject, the frequency spectrum tends to be different depending on the character of the biological tissues on which the ultrasound waves were scanned. That is because, the frequency spectrum has a correlation with the size, the number density, and the acoustic impedance of the scattering body that scatters ultrasound waves. Herein, the term “the character of the biological tissues” implies, for example, a malignant tumor (cancer), a benign tumor, an endocrine tumor, a mucinous tumor, normal tissues, a cyst, or a vascular channel.

The spectrum correction unit 332 uses the reference data corresponding to the subject and corrects each of a plurality of frequency spectrums calculated by the frequency analysis unit 331. In the first embodiment, based on the type of the subject as specified by the user, such as an operator, via the input unit 36, the spectrum correction unit 332 selects the reference data by referring to the storage unit 38. In the first embodiment, the reference data represents a frequency spectrum obtained by analyzing the frequencies of the ultrasound waves obtained from the concerned subject.

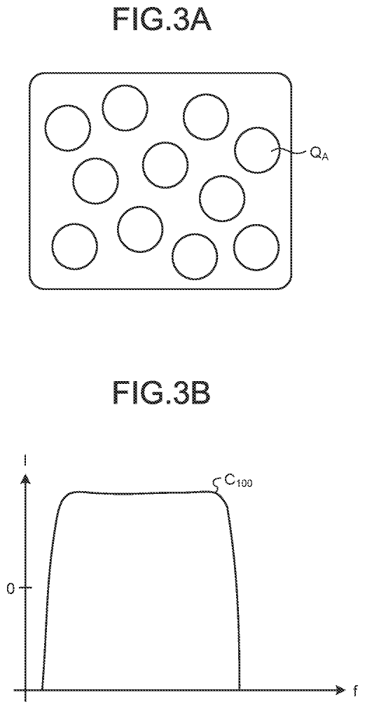

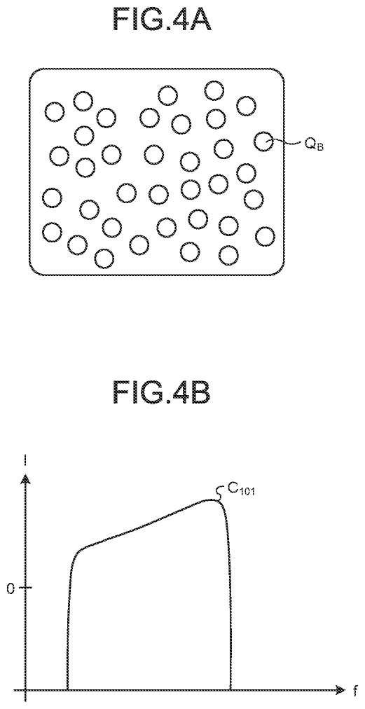

The frequency spectrum of a subject has a different frequency characteristic (waveform) depending on the structure of that object (i.e., the size and the density of the scattering body present in the biological tissues). FIG. 3A is a diagram illustrating an example of the scattering body. FIG. 3B is a diagram for explaining an example of the reference data that is used in the correction operation for correcting the frequency spectrum as performed in the ultrasound imaging apparatus according to the first embodiment; and is a diagram illustrating the reference data based on the ultrasound waves that were scattered or reflected by the scattering body illustrated in FIG. 3A. FIG. 4A is a diagram illustrating an example of the scattering body. FIG. 4B is a diagram for explaining an example of the reference data that is used in the correction operation for correcting the frequency spectrum as performed in the ultrasound imaging apparatus according to the first embodiment; and is a diagram illustrating the reference data based on the ultrasound waves that were scattered or reflected by the scattering body illustrated in FIG. 4A. FIGS. 3A and 4A are diagrams that schematically illustrate portions of mutually different biological tissues. The scattering body Q_{A} illustrated in FIG. 3A and a scattering body Qs illustrated in FIG. 4A are scattering bodies present in mutually different biological tissues (organs) and have mutually different sizes and densities. A frequency spectrum C₁₀₀ illustrated in FIG. 3B is a frequency spectrum based on the ultrasound waves coming from the scattering body Q_{A} illustrated in FIG. 3A. A frequency spectrum C₁₀₁ illustrated in FIG. 4B is a frequency spectrum based on the ultrasound waves coming from the scattering body Q_{B} illustrated in FIG. 4A. Herein, the reference spectrums C₁₀₀ and C₁₀₁ are frequency spectrums obtained as a result of analyzing the frequencies of the ultrasound waves scattered by the scattering bodies of the biological tissues in the normal state.

In FIGS. 3B and 4B, the horizontal axis represents the frequency f. Moreover, in FIGS. 3B and 4B, the vertical axis represents the common logarithm (digital expression) I.

For example, if the reference spectrum C₁₀₀ is selected, then the spectrum correction unit 332 subtracts the reference spectrum C₁₀₀ from the frequency spectrum based on the ultrasound waves obtained from the biological tissues of the subject. As a result of the correction performed using a reference spectrum, for example, in the examples illustrated in FIGS. 3B and 4B, the peak of the intensity of the frequency spectrum of normal biological tissues becomes zero regardless of the type of the biological tissues. Meanwhile, instead of performing the subtraction, the frequency spectrum can be corrected by multiplying a coefficient that is set as reference data on a frequency-by-frequency basis.

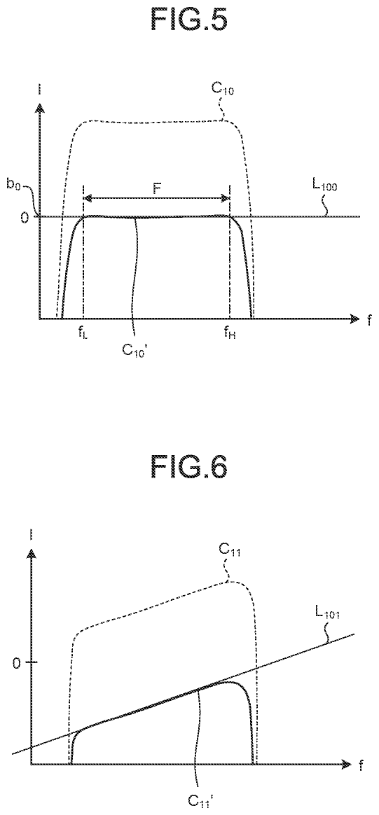

FIG. 5 is a diagram illustrating an example of the post-correction frequency spectrum obtained as a result of the correction performed by the spectrum correction unit 332. In FIG. 5 is illustrated the frequency spectrum that is obtained by correcting the frequency spectrum calculated based on the ultrasound waves coming from the scattering body Q_{A}. In FIG. 5, the horizontal axis represents the frequency f, and the vertical axis represents the common logarithm (digital expression) I. Regarding a straight line L₁₀₀ illustrated in FIG. 5 (hereinafter, called a regression line L₁₀₀), the explanation is given later. The frequency spectrum C₁₀ illustrated using a dashed line in FIG. 5 represents the pre-spectrum-correction frequency spectrum (refer to FIG. 2) that is calculated based on the ultrasound waves coming from the scattering body Q_{A}. That is, a frequency spectrum C₁₀′ is obtained by subtracting the frequency spectrum C₁₀ from the reference data of the corresponding scattering body Q_{A} (for example, reference data C₁₀₀ illustrated in FIG. 3B).

In the frequency spectrum C₁₀′ illustrated in FIG. 5, a lower limit frequency f_{L} and an upper limit frequency f_{H} of the frequency band used in the subsequent computations represent parameters that are decided based on the frequency band of the ultrasound transducer 21 and the frequency band of the pulse signals transmitted by the transceiver 31. With reference to FIG. 5, the frequency band that gets decided by the lower limit frequency f_{L} and the upper limit frequency f_{H} is referred to as a “frequency band F”.

The feature calculation unit 333 calculates, in, for example, the region of interest (ROI) that is set, features of a plurality of frequency spectrums corrected by the spectrum correction unit 332. In the first embodiment, the explanation is given under the premise that two regions of interest having mutually different regions are set. The feature calculation unit 333 includes an approximation unit 333a that approximates a post-correction frequency spectrum by a straight line and calculates the feature of the frequency spectrum before the implementation of attenuation correction (hereinafter, called “pre-correction feature”); and includes an attenuation correction unit 333b that calculates the feature by performing attenuation correction with respect to the pre-correction feature calculated by the approximation unit 333a.

The approximation unit 333a performs regression analysis of a frequency spectrum in a predetermined frequency band, approximates the frequency spectrum by a linear equation (a regression line), and calculates pre-correction features that characterize the approximated linear equation. For example, in the case of the frequency spectrum C₁₀′ illustrated in FIG. 5, the approximation unit 333a performs regression analysis by the frequency band F, approximates the frequency spectrum C₁₀′ by a linear equation, and obtains the regression line L₁₀₀. In other words, the approximation unit 333a calculates the following as the pre-correction features: an inclination a₀ of the regression line L₁₀₀; an intercept b₀ of the regression line L₁₀₀; and a mid-band fit c₀(=a₀f_{M}+b₀) that represents a value on the regression line of a mean frequency f_{M}=(f_{L}+f_{H}) of the frequency band F.

Of the three pre-correction features, the inclination a₀ has a correlation with the size of the scattering body that scatters the ultrasound waves, and is believed to generally have the value in inverse proportion to the size of the scattering body. The intercept b₀ has a correlation with the size of the scattering body, the difference in the acoustic impedance, and the number density (concentration) of the scattering body. More particularly, the intercept b₀ is believed to have the value in proportion to the size of the scattering body, in proportion to the difference in the acoustic impedance, and in proportion to the number density of the scattering body. The mid-band fit c₀ is an indirect parameter derived from the inclination a₀ and the intercept b₀, and provides the intensity of the spectrum in the center of the effective frequency band. For that reason, the mid-band fit c₀ is believed to have a correlation with the size of the scattering body, the difference in the acoustic impedance, and the number density of the scattering body; as well as have a correlation of some level with the luminance of B-mode images. Meanwhile, the feature calculation unit 333 can approximate the frequency spectrums by polynomial equations of two or more dimensions by performing regression analysis.

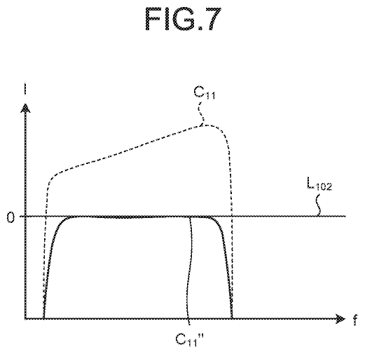

Explained below with reference to FIGS. 5 to 7 are the differences between the regression lines attributed to the reference data that is used. FIG. 6 is a diagram for explaining about the post-correction frequency spectrum obtained by performing correction using reference data that does not correspond to any scattering body. FIG. 7 is a diagram for explaining about the post-correction frequency spectrum obtained by performing correction using reference data that corresponds to a scattering body. With reference to FIGS. 6 and 7, a frequency spectrum Cu illustrated using a dashed line is a frequency spectrum calculated based on the ultrasound waves coming from the scattering body Q.

When the frequency spectrum C₁₀ is subtracted from the reference data of the corresponding scattering body Q_{A} (for example, from the reference data C₁₀₀ illustrated in FIG. 3B), the frequency spectrum C₁₀′ illustrated in FIG. 5 is obtained as explained earlier. Then, the post-correction frequency spectrum C₁₀′ is approximated by a linear equation (a regression line) and the regression line L₁₀₀ is obtained.

On the other hand, if the frequency spectrum C₁₁ is subtracted from the reference data of the non-corresponding scattering body Q_{A} (for example, from the reference data C₁₀₀ illustrated in FIG. 3B), a frequency spectrum C₁₁′ illustrated in FIG. 6 is obtained. Then, the post-correction frequency spectrum C₁₁′ is approximated by a linear equation (a regression line) and a regression line L₁₀₁ is obtained.

Alternatively, if the frequency spectrum C₁₁ is subtracted from the reference data of the corresponding scattering body Q_{B} (for example, from reference data C₁₀₁ illustrated in FIG. 4B), a frequency spectrum C₁₁″ illustrated in FIG. 7 is obtained. Then, the post-correction frequency spectrum C₁₁″ is approximated by a linear equation (a regression line) and a regression line L₁₀₂ is obtained.

If the regression lines L₁₀₀, L₁₀₁, and L₁₀₂ are compared, the regression lines L₁₀₀ and L₁₀₂ that are corrected using the reference data of the respective corresponding scattering bodies theoretically have the same inclination, the same intercept, and the same mid-band fit. On the other hand, the regression line L₁₀₁ that is corrected using the reference data of a non-corresponding scattering body has a different inclination, a different inclination, and a different mid-band fit than the regression lines L₁₀₀ and L₁₀₂. Thus, if the same reference data is used with respect to different biological tissues, then the regression lines obtained from the frequency spectrum of normal biological tissues happen to be different, and the features calculated from such regression lines also happen to differ.

Even when two normal biological tissues are present, if the respective inclinations, intercepts, and mid-band fits are different, then the determination criterion for determining normality or abnormality differs for the features. In that case, either the determination criterion needs to be provided for each frequency spectrum (herein, for the frequency spectrums C₁₀′ and C₁₁′), or the user needs to perform the diagnosis on the screen based on different determination criteria.

In the first embodiment, since the frequency spectrums are corrected using the reference data of the respective corresponding scattering bodies, even if a frequency spectrum is based on the ultrasound waves scattered by a different scattering body, it becomes possible to obtain the features that are diagnosable according to the same determination criterion.

The attenuation correction unit 333b performs attenuation correction with respect to the pre-correction features obtained by the approximation unit 333a. The attenuation correction unit 333b performs attenuation correction with respect to the pre-correction features according to an attenuation ratio. As a result of performing attenuation correction, the features (for example, the inclination a, the intercept b, and the mid-band fit c) are obtained.

The image processing unit 34 includes: a B-mode image data generation unit 341 that generates ultrasound wave image data (hereinafter, called B-mode image data) for displaying an image by converting the amplitude of the echo signals into luminance; and a feature image data generation unit 342 that associates the features, which are calculated by the attenuation correction unit 333b, with visual information and generates feature image data to be displayed along with the B-mode image data. The image processing unit 34 is configured using a general-purpose processor such as a CPU; or using a dedicated processor in the form of an arithmetic circuit such as an FPGA or an ASIC having specific functions.

The B-mode image data generation unit 341 performs signal processing according to known technologies, such gain processing, contrast processing, and y correction, with respect to the B-mode reception data received from the signal processing unit 32; and generates B-mode image data by performing thinning of the data according to the data step width that is decided depending on the display range for images in the display device 4. A B-mode image is a greyscale image having matching values of R (red), G (green), and B (blue), which represent the variables in the case of adapting the RGB color system as the color space.

The B-mode image data generation unit 341 performs coordinate conversion for rearranging the sets of B-mode reception data, which are received from the signal processing unit 32, in order to ensure spatially correct expression of the scanning range in the B-mode reception data; performs interpolation among the sets of B-mode reception data so as to fill the gaps therebetween; and generates B-mode image data. Then, the B-mode image data generation unit 341 outputs the generated B-mode image data to the feature image data generation unit 342.

The feature image data generation unit 342 superimposes visual information, which is associated to the features calculated by the feature calculation unit 333, onto the image pixels in the B-mode image data, and generates feature image data. The feature image data generation unit 342 assigns the visual information that corresponds to the features of the frequency spectrums. For example, the feature image data generation unit 342 associates a hue as the visual information to either one of the inclination, the intercept, and the mid-band fit; and generates a feature image. Apart from the hue, examples of the visual information associated to the features include the color saturation, the brightness, the luminance value, and a color space variable constituting a predetermined color system such as R (red), G (green), and B (blue).

The region-of-interest setting unit 35 sets a region of interest with respect to a data group according to a preset condition or according to the input of an instruction received by the input unit 36. This data group corresponds to the scanning plane of the ultrasound transducer 21. That is, the data group is a set of points (data) obtained from each position of the scanning plane, and each point in the set is positioned on a predetermined plane corresponding to the scanning plane. The region of interest is meant for calculating the features. The size of the region of interest is set, for example, according to the size of the pixels. The region-of-interest setting unit 35 is configured using a general-purpose processor such as a CPU; or using a dedicated processor in the form of an arithmetic circuit such as an FPGA or an ASIC having specific functions.

Herein, based on the setting input (instruction points) that is input via the input unit 36, the region-of-interest setting unit 35 sets the region of interest for calculating the features. The region-of-interest setting unit 35 can place a frame, which has a preset shape, based on the positions of the instruction points; or can form a frame by joining the point groups of a plurality of input points.

The control unit 37 is configured using a general-purpose processor such as a CPU; or using a dedicated processor in the form of an arithmetic circuit such as an FPGA or an ASIC having specific functions. The control unit 37 reads information stored in the storage unit 38, performs a variety of arithmetic processing related to the operating method of the ultrasound imaging apparatus 3, and comprehensively controls the ultrasound imaging apparatus 3. Meanwhile, the control unit 37 can be configured using a common CPU shared with the signal processing unit 32 and the operating unit 33.

The storage unit 38 is used to store a plurality of features calculated for each frequency spectrum by the attenuation correction unit 333b, and to store the image data generated by the image processing unit 34. Moreover, the storage unit 38 includes a reference data storing unit 381 that is used to store the reference data.

Moreover, in addition to storing the abovementioned information, the storage unit 38 is used to store, for example, the information required in amplification (the relationship between the amplification factor and the reception depth), the information required in amplification correction (the relationship between the amplification factor and the reception depth), the information required in attenuation correction, and the information about the window function (such as Hamming, Hanning, or Blackman) required in frequency analysis.

Furthermore, the storage unit 38 is used to store various computer programs including an operating program that is meant for implementing an operating method of the ultrasound imaging apparatus 3. The operating program can be recorded, for distribution purposes, in a computer-readable recording medium such as a hard disc, a flash memory, a CD-ROM, a DVD-ROM, or a flexible disc. Meanwhile, the various computer programs can be made downloadable via a communication network. The communication network is implemented using, for example, an existing public line network, or a local area network (LAN), or a wide area network (WAN); and can be a wired network or a wireless network.

The storage unit 38 having the abovementioned configuration is implemented using a read only memory (ROM) in which various computer programs are installed in advance, and a random access memory (RAM) that is used to store operation parameters and data of various operations.

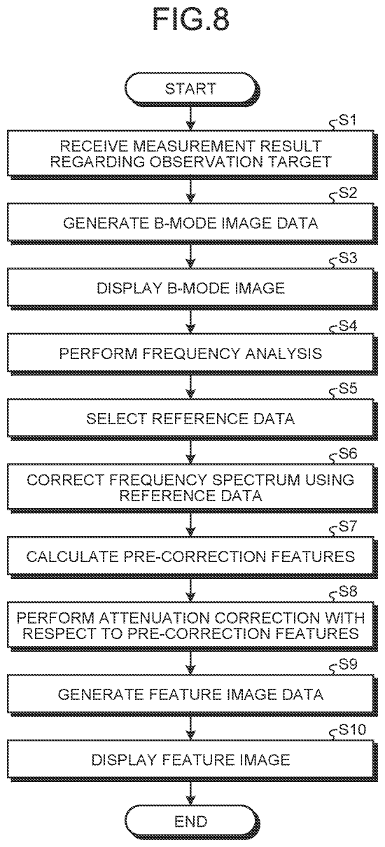

FIG. 8 is a flowchart for explaining an overview of the operations performed by the ultrasound imaging apparatus 3 having the configuration explained above. Firstly, the ultrasound imaging apparatus 3 receives, from the ultrasound endoscope 2, the echo signals representing the measurement result regarding the observation target as obtained by the ultrasound transducer 21 (Step S1).

Then, the B-mode image data generation unit 341 generates B-mode image data using the echo signals received by the transceiver 31, and outputs the B-mode image data to the display device 4 (Step S2). Upon receiving the B-mode image data, the display device 4 displays a B-mode image corresponding to the B-mode image data (Step S3).

Subsequently, the frequency analysis unit 331 performs FFT-based frequency analysis and calculates the frequency spectrums with respect to all sample data groups (Step S4). The frequency analysis unit 331 performs FFT for a plurality of times with respect to each sound ray in the target region for analysis. The result of FFT is stored in the storage unit 38 along with the reception depth and the reception direction.

Meanwhile, at Step S4, the frequency analysis unit 331 either can perform frequency analysis with respect to all regions that received ultrasound signals, or can perform frequency analysis only in the region of interest that is set.

Subsequent to the frequency analysis performed at Step S4, the spectrum correction unit 332 corrects the calculated frequency spectrum (Steps S5 and S6).

Firstly, the spectrum correction unit 332 refers to the reference data storing unit 381 and selects the reference data corresponding to the type of the subject (for example, the biological tissues) specified by the user (Step S5).

The spectrum correction unit 332 uses the selected reference data and corrects the frequency spectrums calculated at Step S4 (Step S6). The spectrum correction unit 332 corrects the frequency spectrums by performing the abovementioned subtraction or by performing coefficient multiplication. As a result of the correction performed by the spectrum correction unit 332, for example, the frequency spectrum C₁₀′ illustrated in FIG. 5 is obtained.

Then, the feature calculation unit 333 calculates the pre-correction features for each post-correction frequency spectrum; performs attenuation correction for eliminating the effect of attenuation of ultrasound waves with respect to the pre-correction features for each frequency spectrum; and calculates the corrected features for each frequency spectrum (Steps S7 and S8).

At Step S7, the approximation unit 333a performs regression analysis of each of a plurality of frequency spectrums generated by the frequency analysis unit 331, and calculates the pre-correction features corresponding to each frequency spectrum (Step S7). More particularly, the approximation unit 333a performs approximation by a linear equation by performing regression analysis of each frequency spectrum, and calculates the inclination a₀, the intercept b₀, and the mid-band fit c₀ as the pre-correction features. For example, the regression line L₁₀₀ illustrated in FIG. 5 is obtained by the approximation unit 333a by performing approximation using regression analysis with respect to the frequency spectrum C₁₀′ of the frequency band F.

Then, with respect to the pre-correction features obtained by the approximation unit 333a by approximation with respect to each frequency spectrum, the attenuation correction unit 333b performs attenuation correction using an attenuation rate, calculates corrected features, and stores them in the storage unit 38 (Step S8).

Subsequently, with respect to each pixel in the B-mode image data generated by the B-mode image data generation unit 341, the feature image data generation unit 342 superimposes visual information, which is associated to the features calculated at Step S8, according to the preset color combination condition; and generates feature image data (Step S9).

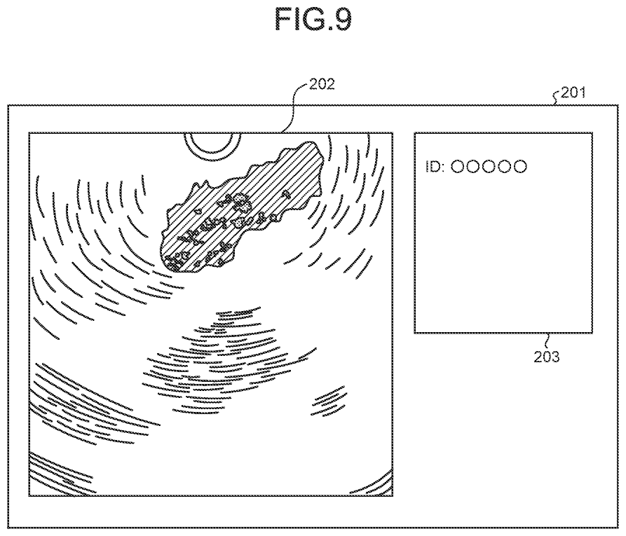

Then, under the control of the control unit 37, the display device 4 displays a feature image corresponding to the feature image data generated by the feature image data generation unit 342 (Step S10). FIG. 9 is a diagram that schematically illustrates an exemplary display of a feature image in the display device 4. A feature image 201 illustrated in FIG. 9 includes a superimposed-image display portion 202 in which an image obtained by superimposing the visual information related to the features on a B-mode image is displayed, and includes an information display portion 203 in which identification information of the observation target (the subject) is displayed.

As described above, in the first embodiment of the disclosure, the reference data corresponding to the type of the biological tissues (for example, the type of the scattering body such as an organ) is kept ready in advance, and the frequency spectrums of the subject are corrected using the reference data. According to the first embodiment, as a result of performing correction using each set of reference data, the intensity of each type of frequency spectrum gets adjusted to a similar spectrum and thus the range gets set for obtaining the features according to the linear approximation performed by the approximation unit 333a. Hence, the characteristics of the subject as obtained from the frequency spectrums can be analyzed in a uniform manner regardless of the type of the subject. For example, even when types of subjects are different, the post-correction frequency spectrums have the same waveform (for example, refer to FIGS. 5 and 7) regardless of the types of biological tissues. As a result, the determination criterion for determining the characteristics of the biological tissues (for example, malignant or benign) becomes the same and the analysis can be performed in a uniform manner. Moreover, even when the subjects having different types are present in the same image, each subject can be analyzed without changing the criteria.

### Second Embodiment

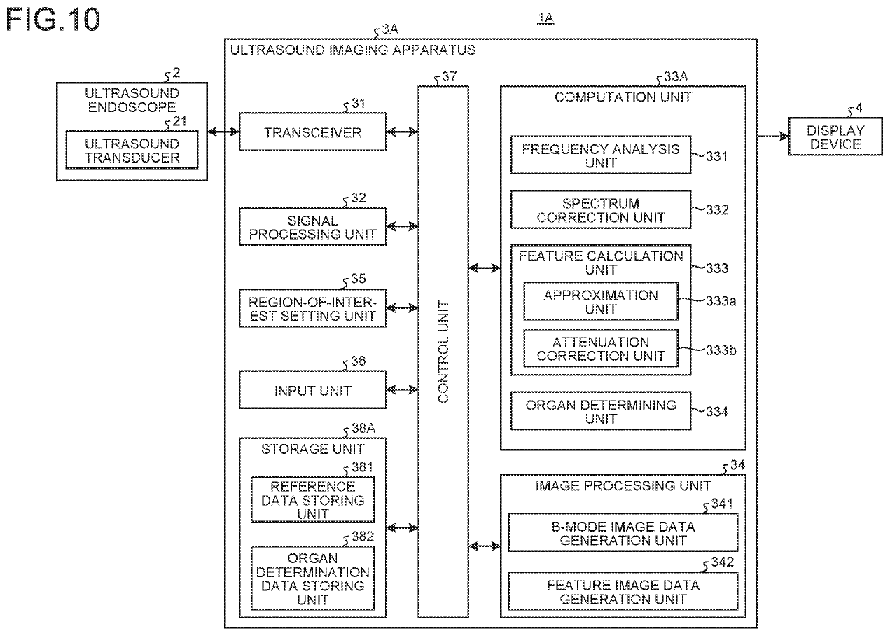

FIG. 10 is a block diagram illustrating a configuration of an ultrasound imaging system 1A that includes an ultrasound imaging apparatus 3A according to a second embodiment of the disclosure. The ultrasound imaging system 1A illustrated in FIG. 10 includes; the ultrasound endoscope 2 (an ultrasound probe) that transmits ultrasound waves to a subject and receives the ultrasound waves reflected from the subject; the ultrasound imaging apparatus 3A that generates ultrasound images based on the ultrasound wave signals obtained by the ultrasound endoscope 2; and the display device 4 that displays the ultrasound images generated by the ultrasound imaging apparatus 3A. In the ultrasound imaging system 1A according to the second embodiment, other than the fact that the ultrasound imaging apparatus 3A is substituted for the ultrasound imaging apparatus 3 of the ultrasound imaging system 1, the configuration is same as the ultrasound imaging system 1. Thus, the following explanation is given only about the ultrasound imaging apparatus 3A that has a different configuration than the first embodiment.

Regarding the ultrasound imaging apparatus 3A, other than the facts that a computation unit 33A is substituted for the computation unit 33 and that a storage unit 38A is substituted for the storage unit 38, the configuration is same as the configuration of the ultrasound imaging apparatus 3. The computation unit 33A includes an organ determining unit 334, in addition to having the same configuration as the computation unit 33. Thus, the following explanation is given only about the operations of the storage unit 38A and the organ determining unit 334 that represent the different configuration than the first embodiment. The organ determining unit 334 is equivalent to a type determining unit.

The storage unit 38A includes an organ determination data storing unit 382, in addition to having the same configuration as the storage unit 38. The organ determination data storing unit 382 is used to store organ determination data, such as spectrum data and the intensity distribution, that enables the organ determining unit 334 to determine the organ.

The organ determining unit 334 refers to the input data and to the organ determination data stored in the organ determination data storing unit 382, and determines the organs included as information in the input data. For example, when a frequency spectrum is input, the organ determining unit 334 refers to the organ determination data storing unit 382; compares the pattern of the input frequency spectrum with the frequency spectrums of various types; and determines the organ. Meanwhile, other than determining the organ according to the frequency spectrum, the organ determining unit 334 can determine the organ also using the values of the B-mode image (the luminance value and the RGB value).

In the second embodiment, the ultrasound imaging apparatus 3A performs operations in an identical manner to the first embodiment (refer to FIG. 8). In the second embodiment, at the time of selecting the reference data at Step S5, the organ determining unit 334 performs an organ determination operation, and the reference data is selected based on the determination result.

In the second embodiment, since the organ is automatically determined and the frequency spectrums are corrected based on the reference data corresponding to the determined organ; a difficult-to-distinguish organ gets appropriately determined and, even if the user is a beginner, the reference data is appropriately selected. In the second embodiment too, the characteristics of the subject as obtained from the frequency spectrums can be analyzed in a uniform manner regardless of the type of the subject.

### First Modification Example of Second Embodiment

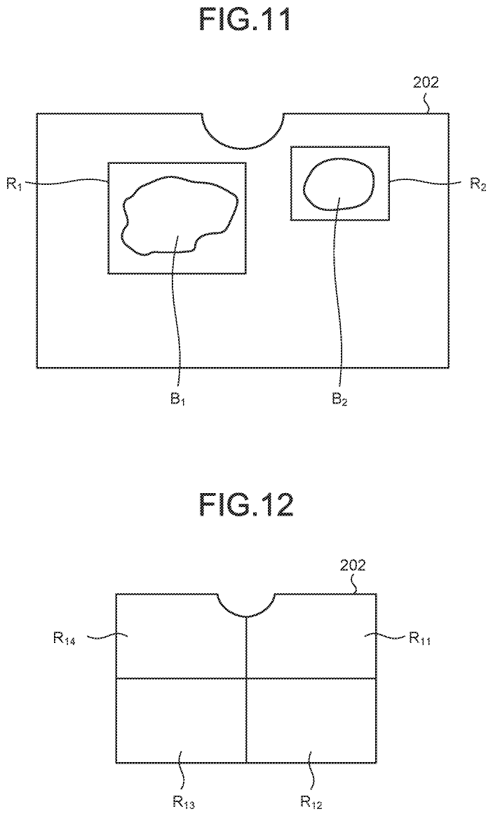

Explained below with reference to FIG. 11 is a first modification example of the second embodiment. FIG. 11 is a diagram for explaining an organ determination operation performed in an ultrasound imaging apparatus according to the first modification example of the second embodiment of the disclosure. Herein, an ultrasound imaging system according to the first modification example has the same configuration as the ultrasound imaging system 1A according to the second embodiment. Hence, that explanation is not given again. Thus, the following explanation is given only about the operations that are different than the operations according to the second embodiment.

In the first modification example, regarding the regions of interest set by the user, organ determination is performed and the reference data is selected. The user sets regions of interest in a B-mode image in which organ determination is to be performed. With reference to FIG. 11, regarding biological tissues B₁ and B₂, regions of interest R₁ and R₂ surrounding them are respectively set.

The organ determining unit 334 determines the organs present in the regions of interest that are set (i.e., in the regions of interest R₁ and R₂). In an identical manner to the second embodiment, the organ determining unit 334 refers to the organ determination data storing unit 382 and determines the organs. Then, the spectrum correction unit 332 selects the reference data corresponding to the organ determined in each of the regions of interest R₁ and R₂, and uses the reference data in correcting the frequency spectrums calculated in the corresponding region of interest. Subsequently, in an identical manner to the first embodiment, the feature calculation unit 333 calculates the features.

In the first modification example, even when different types of biological tissues are captured in the same display image, organ determination can be performed and appropriate sets of reference data can be selected. According to the first modification example, even when different types of biological tissues are captured in the same display image, the characteristics of the subject as obtained from the frequency spectrums can be analyzed in a uniform manner regardless of the type of the subject. Meanwhile, if the reference data is same regardless of the types of the biological tissues, there are different criteria for each biological tissue and the user needs to determine about malignancy and benignity while changing the criteria in the head. However, in the first modification example, since the criteria are consolidated, malignancy and benignity can be determined without having to change the criteria.

Meanwhile, in the first modification example, in addition to the organ determination performed by the organ determining unit 334; the input unit 36 can receive, for each region of interest, information about the type of the organ (the type of the observation target) present in that region of interest, and the spectrum correction unit 332 can select the reference data according to the received information and correct the frequency spectrums. In that case, the configuration is same as the ultrasound imaging system 1 according to the first embodiment.

### Second Modification Example of Second Embodiment

Explained below with reference to FIG. 12 is a second modification example of the second embodiment. FIG. 12 is a diagram for explaining an organ determination operation performed in an ultrasound imaging apparatus according to the second modification example of the second embodiment of the disclosure. Herein, an ultrasound imaging system according to the second modification example has the same configuration as the ultrasound imaging system 1A according to the second embodiment. Hence, that explanation is not given again. Thus, the following explanation is given only about the operations that are different than the operations according to the second embodiment.

In the second modification example, a B-mode image is divided into regions; organ determination is performed for each divided region; and the corresponding reference data is selected. At that time, the user inputs, for example, the number of divisions of the B-mode image. In FIG. 12 is illustrated an example in which the B-mode image is divided into four regions.

The organ determining unit 334 determines the organ captured in each divided region (i.e., each of divided regions R₁₁ to R₁₄). In an identical manner to the second embodiment, the organ determining unit 334 refers to the organ determination data storing unit 382 and determines the organ captured in each divided region. The spectrum correction unit 332 selects the reference data corresponding to the organ determined in each divided region, and uses it to correct the frequency spectrums calculated in that divided region. Then, the feature calculation unit 333 calculates the features in an identical manner to the first embodiment.

In the second modification example, even when different types of biological tissues are captured in the same display image, organ determination can be performed for each divided region and appropriate reference data can be selected. According to the second embodiment, even when different types of biological tissues are captured in the same display image, the characteristics of the subject as obtained from the frequency spectrums can be analyzed in a uniform manner regardless of the type of the subject.

Meanwhile, in the second modification example, although a B-mode image is divided into four regions, the number of divisions can be different than four. In order to enhance the accuracy of organ determination, it is desirable to increase the number of divisions and to perform organ determination in more detail. Moreover, in the second modification example, the B-mode is assumed to have a rectangular outer rim. However, alternatively, the B-mode image can have a fan shape in tune with the scanning area of ultrasound waves, and that B-mode image can be divided into regions.

Although the embodiments of the disclosure are described above, the disclosure is not limited by those embodiments. That is, the disclosure can be construed as embodying all modifications such as other embodiments, additions, alternative constructions, and deletions that may occur to one skilled in the art that fairly fall within the basic teaching herein set forth. In the first and second embodiments described above, as the ultrasound probe, it is possible to use an external ultrasound probe that radiates ultrasound waves from the body surface of the subject. Usually, an external ultrasound probe is used in observing an abdominal organ (the liver, the gallbladder, or the urinary bladder), the breast mass (particularly the lacteal gland), and the thyroid gland.

Thus, the ultrasound imaging apparatus, the operating method of the ultrasound imaging apparatus, and the operating program for the ultrasound imaging apparatus are suitable in analyzing the characteristics of the observation target, which are obtained from the frequency spectrums, in a uniform manner regardless of the type of the observation target.

According to the disclosure, it becomes possible to analyze the characteristics of the observation target, which are obtained from the frequency spectrums, in a uniform manner regardless of the type of the observation target.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging apparatus comprising:
a processor; and
a storage,
the storage being configured to store first-type reference data corresponding to a first observation target, and second-type reference data corresponding to a second observation target, and
the processor being configured to
transmit, to an ultrasound probe, a signal for making the ultrasound probe transmit an ultrasound wave to an observation target,
receive an echo signal representing an electrical signal obtained by conversion of an ultrasound wave received by the ultrasound probe,
perform frequency analysis based on the echo signal to calculate a frequency spectrum,
obtain reference data,
correct the frequency spectrum using the reference data,
calculate a feature based on the corrected frequency spectrum,
when the observation target is the first observation target, obtain the first-type reference data as the reference data, and
when the observation target is the second observation target, obtain the second-type reference data as the reference data.

2. The ultrasound imaging apparatus according to claim 1, further comprising an input device configured to receive input of type of the observation target, wherein
the processor is further configured to select reference data corresponding to type of the observation target as received by the input device, and correct the frequency spectrum using the selected reference data.

3. The ultrasound imaging apparatus according to claim 2, wherein
when a plurality of regions of interest is set, the input device is further configured to receive input of type of observation target for each region of interest, and
the processor is further configured to correct the frequency spectrum using reference data selected for each region of interest.

4. The ultrasound imaging apparatus according to claim 1, wherein
the storage is further configured to store determination data meant for determining type of the observation target, and
the processor is further configured to
determine type of the observation target based on the echo signal and the determination data, and
select reference data of type corresponding to result of the determination, and
correct the frequency spectrum using the selected reference data.

5. The ultrasound imaging apparatus according to claim 4, wherein the processor is further configured to
generate ultrasound image data for displaying an image by converting amplitude of the echo signal into luminance,
set a plurality of regions of interest with respect to an ultrasound image corresponding to the ultrasound image data,
perform determination of type of observation target included in each of the set plurality of the regions of interest,
select, for each region of interest, reference data of type corresponding to result of the determination, and
correct the frequency spectrum using the selected reference data.

6. The ultrasound imaging apparatus according to claim 4, wherein the processor is further configured to
generate ultrasound image data for displaying an image by converting amplitude of the echo signal into luminance,
perform determination of type of each observation target using the frequency spectrum or the luminance,
select, for each region of interest, reference data of type corresponding to result of the determination, and
correct the frequency spectrum using the selected reference data.

7. The ultrasound imaging apparatus according to claim 4, wherein the processor
generate ultrasound image data for displaying an image by converting amplitude of the echo signal into luminance,
divide an ultrasound image corresponding to the ultrasound image data into regions,
perform determination of type of observation target included in each divided region of the ultrasound image,
select, for each divided region, reference data of type corresponding to result of the determination, and
correct the frequency spectrum using the selected reference data.

8. The ultrasound imaging apparatus according to claim 1, wherein the reference data is a reference spectrum corresponding to type of biological tissue.

9. An operating method of an ultrasound imaging apparatus configured to generate an ultrasound image based on an ultrasound signal obtained by an ultrasound probe which includes an ultrasound transducer for transmitting an ultrasound wave to an observation target and receiving an ultrasound wave reflected from the observation target, the operating method comprising:
transmitting, to the ultrasound probe, a signal for making the ultrasound probe transmit an ultrasound wave to an observation target,
receiving an echo signal representing an electrical signal obtained by conversion of an ultrasound wave received by the ultrasound probe;
performing frequency analysis based on the echo signal to calculate a frequency spectrum;
obtaining first-type reference data when the observation target is a first observation target,
obtaining second-type reference data when the observation target is a second observation target,
correcting the frequency spectrum using the obtained first-type reference data or the obtained second-type reference data; and
calculating a feature based on the corrected frequency spectrum.

10. A non-transitory computer-readable recording medium with an executable program stored thereon, the program causing an ultrasound imaging apparatus configured to generate an ultrasound image based on an ultrasound signal obtained by an ultrasound probe which includes an ultrasound transducer for transmitting an ultrasound wave to an observation target and receiving an ultrasound wave reflected from the observation target, to execute:
transmitting, to the ultrasound probe, a signal for making the ultrasound probe transmit an ultrasound wave to an observation target,
receiving an echo signal representing an electrical signal obtained by conversion of an ultrasound wave received by the ultrasound probe;
performing frequency analysis based on the echo signal to calculate a frequency spectrum;
obtaining first-type reference data when the observation target is a first observation target,
obtaining second-type reference data when the observation target is a second observation target,
correcting the frequency spectrum using the obtained first-type reference data or the obtained second-type reference data; and
calculating a feature based on the corrected frequency spectrum.
